(19) United States
(12) Patent Application Publication
(10) Pub. No.: US 2025/0049384 A1
(43) Pub. Date: Feb. 13, 2025

(54) PHOTO ACOUSTIC QUALITATIVE ULTRASOUND BONE DENSITOMETER

(71) Applicants: The Regent of the University of Michigan, Ann Arbor, MI (US); IF, LLC, Stoughton, WI (US)

(72) Inventors: Richard Franklin Morris, Stoughton, WI (US); Xueding Wang, Ann Arbor, MI (US); Richard A. Hogle, Ann Arbor, MI (US); Duane A. Kaufman, Ann Arbor, MI (US); Ken Kozloff, Ann Arbor, MI (US)

(21) Appl. No.: 18/446,286

(22) Filed: Aug. 8, 2023

(51) Int. Cl. A61B 5/00

(52) U.S. Cl. CPC A61B 5/4509; A61B 5/0095; A61B 5/4595; A61B 5/702

(57) ABSTRACT

An ultrasound bone densitometer employs transmitted ultrasound and laser stimulated ultrasound to provide a more complete measure of bone health. Sources of error including secondary laser stimulation, signal dependency on direction of laser light travel, registration, and signal chain noise are addressed in the construction of the densitometer.

### STATEMENT REGARDING FEDERALLY SPONSORED RESEARCH OR DEVELOPMENT

--

### CROSS REFERENCE TO RELATED APPLICATION

--

### Background of the Invention

The present invention relates to bone densitometers and in particular to an ultrasound bone densitometer providing improved bone density measurements through combined transmitted and photo stimulated ultrasound.

Bone densitometers provide noninvasive measurements of bone density and structure and are useful for assessing loss of bone mineralization (osteoporosis), as can occur with aging. One common type of bone densitometer employs dual energy x-ray absorptiometry (DEXA) to image bone isolated from soft tissue and to provide quantitative measurements of bone mineral density (BMD) typically in the spine and hip. DEXA bone densitometers are considered the gold standard for bone densitometry but are unable to assess important parameters such as bone microarchitecture, bone elastic properties, and chemical information also important in determining fracture risk.

Quantitative ultrasound (QUS) bone density measurements provide an alternative to DEXA that avoids the use of ionizing radiation and has a lower cost making bone density screening practical. QUS densitometers make measurements of bone structures which are readily accessible to the imaging technique. QUS densitometers may use opposed ultrasound transducers operating to transmit an ultrasound pulse from one side of the bone to the other side, where the pulse is measured to deduce time-of-flight (TOF) and broadband ultrasound attenuation (BUA), the latter indicating change in attenuation as a function of ultrasound frequency. Such measurements correlate with bone density but are less successful in measuring the other dimensions of fracture risk described above. One such measurement site is the os calcis (heel bone) which is readily accessible and provides a good proxy for bone health because of its weight-bearing nature. Another such measurement site is the distal radius (forearm) which is also readily accessible and is a high site of high risk for skeletal fracture. Broadly speaking, QUS densitometers can be applied to any skeletal site in which adequate signal can be transmitted and received.

Recently, laser-induced photo acoustic (PA) ultrasound has been proposed to augment QUS by characterizing the chemical components and microarchitecture of the os calcis. The laser pulse excites broad regions of the bone tissue simultaneously allowing separation of trabecular signals characterizing spongy interior trabecular bone from cortical signals characterizing outer cortical bone by differences in time-of-flight. Distinguishing these two bone types improves sensitivity to changes in the trabecular bone, which is more sensitive to bone loss, and thus characterizes microarchitecture. The frequency of the laser inducing the ultrasound also allows non-organic minerals such as hydroxyapatite to be distinguished from organic materials such as lipids and hemoglobin, providing insight into chemical composition of the bone marrow, and possibly organic materials such as collagen or other non-collagenous proteins, providing insight into the chemical composition of the bone matrix itself.

PA-assisted bone densitometry is described in Ting Feng, et al, Functional Photoacoustic and Ultrasonic Assessment of Osteoporosis: A Clinical Feasibility Study, AAAS BME Frontiers, volume 2020, article ID 1081540 27-10-2020, describing research conducted by coinventors of the present application and hereby incorporated by reference.

Practical implementation of this PA-assisted bone densitometry faces significant challenges because of the challenge in the delivery of light and ultrasound through bone and detection of the resulting faint ultrasound signal.

### SUMMARY OF THE INVENTION

The present inventors have determined that photo stimulation of ancillary tissue and environmental materials can significantly affect the quality of the photo acoustic measurement. The present invention addresses this through a number of innovations including, in some embodiments, a movable light source that can be inserted between the patient and transducer used for QUS providing improved optical coupling free from interference by ultrasound coupling associated with the QUS transmitting transducer. An ankle stop preserves foot position and patient registration when the light source is in position and the surrounding surfaces are treated to be light absorbing.

The inventors have also determined that the PA measurement is directionally dependent and accordingly, in some embodiments, acquires and combines PA measurements taken in opposed directions. Separate signal and amplification chains allow transmitted and induced ultrasound signals to be optimally processed by adjusting signal bandwidth.

Specifically, in one embodiment, the invention provides an ultrasound bone densitometer having a first and second laterally opposed ultrasound transducer for transmitting an ultrasound signal through a bone. A light source is movable between a first position in a region between the bone and the second opposed ultrasound transducer, and a second position displaced from the region between the bone and the second opposed ultrasound transducer, the light source operating in the first position to direct the light beam along an axis through the bone. A controller controls movement of the light source and activation of the light source and second ultrasound transducer and collects data from the first ultrasound transducer.

It is thus a feature of at least one embodiment of the invention to allow capture of QUS and PA measurements in a single clinical instrument with improved coupling of light to the bone.

The light source maybe a diverter receiving off-axis light from a laser to divert the light along the axis laterally through the bone.

It is thus a feature of at least one embodiment of the invention to permit the introduction of a high-intensity light source in a narrow space between an ultrasound transducer and the bone.

The diverter may be a front surface mirror sealed within a liquid tight housing to redirect the light beam through a sealed cavity window through the bone.

It is thus a feature of at least one embodiment of the invention to provide a robust mechanism for diverting a laser beam received off axis and that is resistant to environmental damage.

The ultrasound bone densitometer may further include first and second inflatable coupling membranes positioned between the first and second opposed transducers and respective sides of the bone wherein the inflatable coupling membrane for the second opposed ultrasound transducer is controllably deflated by the controller to permit movement of the laser diverter to the first position.

It is thus a feature of at least one embodiment of the invention to eliminate interference between coupling media for the ultrasound transducer and the light.

The ultrasound bone densitometer may further include an ankle stop positioned around the second inflatable coupling membrane to support the ankle against lateral movement when the inflatable coupling for the second opposed ultrasound transducer is deflated.

It is thus a feature of at least one embodiment of the invention to maintain registration of the foot between measurements by incident ultrasound and laser-induced stimulation allowing successive measurements to be rapidly acquired without patient repositioning.

The ultrasound bone densitometer may include a housing providing a cavity for receiving and supporting a patient foot therein with the bone positioned between first and second laterally opposed ultrasound transducers at either of two positions having a respective 180° rotation about a vertical axis.

It is thus a feature of at least one embodiment of the invention to permit simple, bidirectional measurements through the ankle without duplication of transducers and with light sources on two opposite sides of the ankle within the housing.

The inner surfaces of the cavity may provide a reflectivity of less than 20% in the range of 690-950 nm.

It is thus a feature of at least one embodiment of the invention to minimize interference from reflected light stimulating ultrasound from tissue and materials outside of the bone being investigated.

The ultrasound bone densitometer may further include a light-blocking cover fitting over the cavity during activation of the laser.

It is thus a feature of at least one embodiment of the invention to provide additional patient safety by blocking laser light which may escape from the cavity.

The light-blocking cover may be an inflatable light baffle extended over the foot by inflation.

It is thus a feature of at least one embodiment of the invention to provide an automated light-blocking system.

The ultrasound bone densitometer may further include a first and second amplifier path for receiving signals from the first ultrasound transducer, the first amplifier path receiving signals during activation of the second ultrasound transducer and the second amplifier path receiving signals during activation of the laser; wherein the first amplifier path has a greater bandwidth than the second amplifier path.

It is thus a feature of at least one embodiment of the invention to exploit the narrow bandwidth of the PA ultrasound to boost signal-to-noise ratio.

The controller may operate to measure ultrasound signals produced by the laser and captured by the first ultrasound transducer when the laser is both directed from a left to right direction and right to left direction.

It is thus a feature of at least one embodiment of the invention to provide improved PA measurements by combining bilateral signal capture.

These particular objects and advantages may apply to only some embodiments falling within the claims and thus do not define the scope of the invention.

### BRIEF DESCRIPTION OF THE DRAWINGS

FIG. 1 is a perspective view of an ultrasound bone densitometer constructed according to one embodiment of the invention and positioned to receive a patient's foot in either of two rotated positions;

FIG. 2 is a fragmentary perspective view similar to FIG. 1 showing inflation of light baffles and a positioning of opposed ankle braces to alternately support the ankle or prevent the escape of light from the cavity;

FIG. 3 is a top plan fragmentary view of internal opposed ultrasound transducers in the first configuration for making a QUS measurement;

FIG. 4 is a side elevational view of a second opposed ultrasound transducer showing a light source introduced in front of that transducer and the surrounding ankle support ring;

FIG. 5 is a figure similar to FIG. 3 showing the light source as positioned for PA measurement;

FIG. 6 is a front elevational view of an ultrasound array transducer for receiving ultrasound signals from an opposed ultrasound transducer or from light-stimulated tissue;

FIG. 7 is a top plan view of the light source of FIGS. 3-5 in cross section showing an internal first surface mirror within a sealed housing; and

FIG. 8 is a schematic block diagram of the components of the invention communicating with a controller for control thereof according to a stored program.

### DETAILED DESCRIPTION OF THE PREFERRED EMBODIMENT

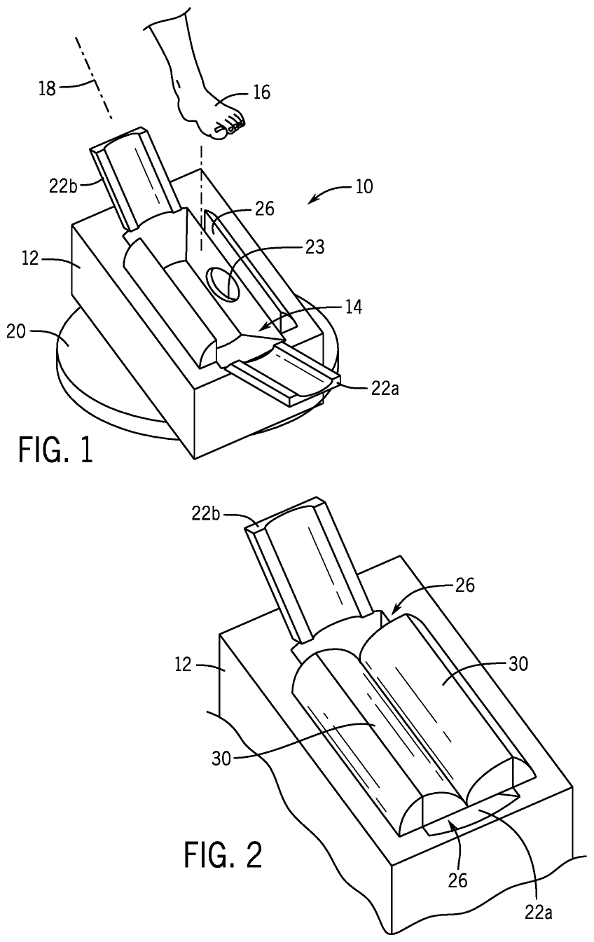

Referring now to FIG. 1, a dual-mode ultrasound bone densitometer 10 may provide a housing 12 having an upwardly open cavity 14 that may receive a patient's foot 16 downwardly therein with the patient's leg extending generally along a longitudinal axis 18. The housing 12 may be mounted on a turntable 20 so that it may be rotated 180° about a vertical axis to either of two positions allowing the patient's foot 16 to be received in the open cavity 14 in either rotated position of the housing 12 without relocation of the patient. As will be discussed below, this rotation of the housing 12 on the turntable 20 allows measurements of the patient's foot to be made using photo acoustic stimulation with light passing both from the left to the right side of the patient's foot and from the right to the left side of the patient's foot from a single laser source.

Referring still to FIG. 1, laterally opposite ends of the cavity 14 may have pivoting calf rests 22a and 22b moving between an opened position (as shown) extending upward and laterally away from the cavity 14 at about 45° to provide a support for the patient's calf when the patient's foot 16 is inserted into the cavity 14 (for when the calf rest 22 is closest to the patient), and folding to a closed horizontal position over the cavity 14 to cover the patient's foot 16 and block escaping laser light (for when the calf rest 22 is furthest from the patient).

Referring also to FIG. 2, transversely opposed, and longitudinally extending openings 26 may flank the upper edge of the cavity 14 to release inflatable light baffles 30 to extend over the cavity 14 to absorb light passing out of the cavity 14 during operation of the dual-mode ultrasound bone densitometer 10. The light baffles 30 flexibly conform to the patient's foot 16 sealing about the ankle (not shown but positioned on calf rest 22b) and may inflate over the top of the calf rest 22a when that calf rest 22a is in the closed position to further prevent the escape of laser light from the cavity 14.

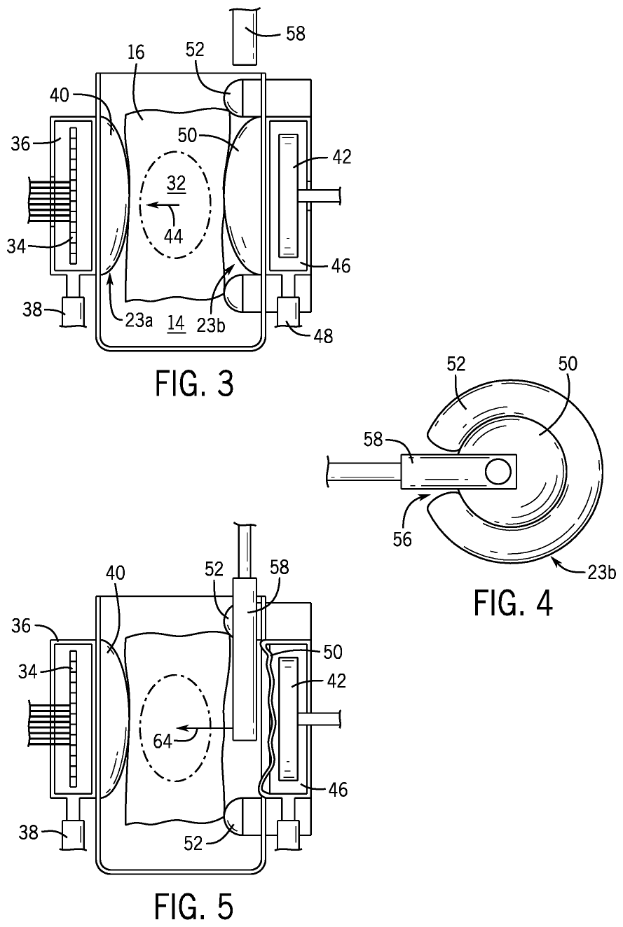

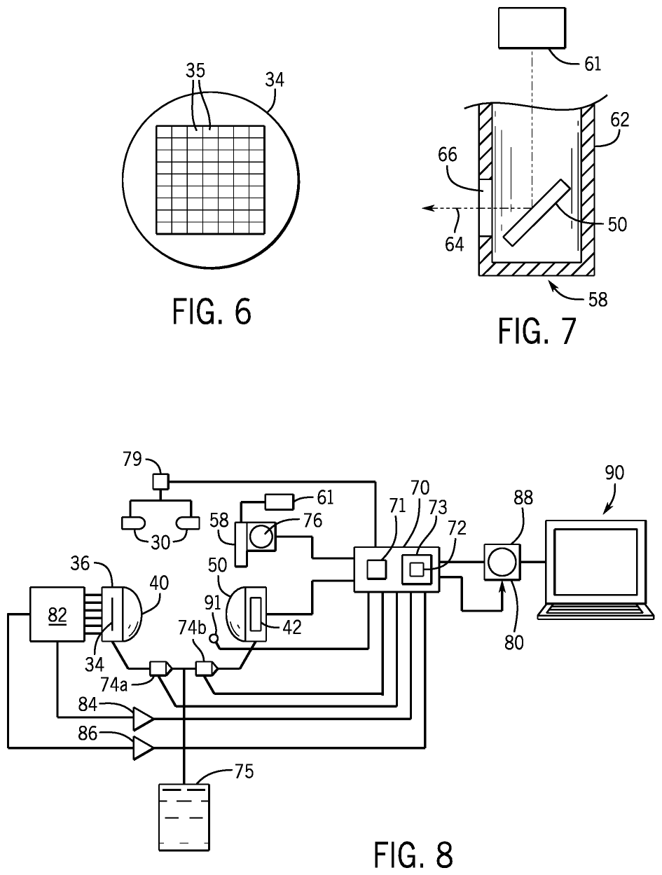

Referring now to FIGS. 1 and 3, when the patient's foot 16 is inserted into the cavity 14, the cavity features align the os calcis 32 of patient's foot 16 between the transversely opposed two transducer windows 23a and 23b. Referring also to FIG. 6, positioned behind transducer window 23a is an array ultrasound transducer 34 having multiple electrically independent transducer elements 35, for example, arranged in rows and columns, each capable of detecting a received ultrasound signal to produce a corresponding electrical signal. The array ultrasound transducer 34 is held in a chamber 36 that may be filled through a pump inlet 38, for example, with distilled water and distend a flexible membrane 40 forming one side of the cavity 14 to press against a right side of the patient's ankle for good acoustic coupling between the ankle and the array ultrasound transducer 34.

Positioned behind the opposing transducer window 23b is a single element ultrasound transducer 42 for transmitting a uniform beam of ultrasound 44 through the os calcis 32 toward the array ultrasound transducer 34. The single element ultrasound transducer 42 is also held in chamber 46 inflatable with a coupling liquid through pump inlet 48 to extend flexible membrane 50 to press against the left side of the patient's ankle for good acoustic coupling. Ultrasound energy passing from the single element ultrasound transducer 42 to the array ultrasound transducer 34 is used for QUS measurements of the os calcis 32.

Isopropyl alcohol may be applied on the outside of the flexible membranes 40 and 50 to complete the acoustic path to the skin. An array ultrasound transducer 34 suitable for use in this application may be constructed as described in U.S. Pat. No. 6,012,779, hereby incorporated by reference.

Referring now to FIG. 4, at the periphery of the transducer window 23 and partially encircling the membrane 50 is an ankle support ring 52 extending inwardly into the cavity to contact the left side of the patient's ankle to maintain it in a single registration position independent of any flexibility in the membranes 50 and 40 or the deflation of membrane 50 as will now be described.

Referring also to FIG. 5, an opening 56 in a side of the ankle support ring 52 allows lateral insertion of a laser probe 58 into the space between the single element ultrasound transducer 42 and the patient's foot 16 when the membrane 50 is deflated pulling back away from the ankle.

Referring momentarily to FIG. 7, the laser probe 58 may include a diagonally mounted front surface mirror 60 sealed within a housing 62 of the laser probe 58 to project a laser beam 64 through a protective window 66 toward the os calcis 32. The laser beam 64 may pass along the longitudinal horizontal axis through the housing 62 from an offset class IV Nd:YAG tunable laser 61, for example, commercially available under the trade name of Phocus Mobile HE OPOTEK, communicating with the laser probe 68 through a fiber coupling. In one embodiment, the laser 61 may have a continuous tuning range of 690 to 950 nm and the repetition rate of 10 Hz with 5 ns pulse width. The pulse energy of the laser is configured for each programmed wavelength such that the light fluence at the skin is 60 mJ over a 2-cm diameter area on the skin, resulting in a light fluence of 19.1 mJ/cm² below the ANSI Maximum Permissible Exposure limit for Skin Exposure (ANSI Z136.1-2014 Tables 6 & 7). During measurement, the wavelength is stepped by a constant step size of 10 nm to create an absorption frequency profile being a measure of induced ultrasound energy as a function of laser frequency.

The front surface mirror 60 receives the laser beam 64 along the lateral axis before diverting the laser beam 64 by 90° to be directed toward the os calcis 32.

Within this optical spectrum range of 690-950 nm, deoxy-hemoglobin has an absorption peak around 760 nm, oxy-hemoglobin has strong absorption at 900 nm, and lipid has an absorption peak at 930 nm. The optical absorption of hydroxyapatite decreases monotonically in this spectral range. The cumulative effect of light absorption in these tissues in the os calcis can be deconvolved, allowing the induced ultrasound to be used for chemical analysis.

Referring now to FIG. 8, the dual-mode ultrasound bone densitometer 10 may include a controller 70 having one or more processors 71 executing a stored program held in computer memory 73 and controlling the various above-described elements to make successive QUS PA measurements in two directions through the os calcis 32 controlling successive inflation and deflation of the necessary membranes 40 and 50 an introduction and removal of the laser probe 58. In this respect, the controller 70 may communicate with a first and second pump 74a and 74b communicate with a temperature-controlled reservoir 75 of water. Timed control of the pumps 74a and 74b allows for independently inflating and deflating membranes 40 and 50, deflating both membranes before insertion of the foot 16, inflating them for a first QUS measurement, and deflating membrane 50 for insertion of the laser probe 58 by activation of a linear motor 76 for a PA measurement. This is followed by a withdrawal of the probe 58 using the motor 76 and deflation of membrane 40 for removal of the patient's foot and rotation of the dual-mode ultrasound bone densitometer 10 on turntable 20. Proper rotation may be confirmed by a limit switch 80.

The rotation allows membranes 40 and 50 to be re-inflated for a repeat of this process described above with a measurement through an opposite lateral direction through the os calcis 32. The second measurement may make a measurement with the laser only if desired to the extent that the ultrasound measurements tend to be equivalent. Prior to each measurement, the controller 70 may control a pump 79 to inflate and deflate the baffles 30 to cover the patient's foot during activation of the laser 61. Confirmation of this light baffle position may be determined, for example, by a photodetector 91 within the cavity 14 or other interlock system.

The controller 70 may control the timing and wave shape of an ultrasound pulse produced by single element ultrasound transducer 42, and an array ultrasound transducer 34 may detect ultrasonic signals both from the single element ultrasound transducer 42 and from the laser probe 58 at successive times. A multiplexer 82 conducts electrical signals from the array ultrasound transducer 34 to a first low-noise amplifier 84 having broad band frequency characteristics for measurement of the ultrasound energy from the single element ultrasound transducer 42 during QUS measurements. During PA measurements, electrical signals from the array ultrasound transducer 34 are routed to a low-noise narrowband amplifier 86. In one example, amplifier 84 may have a frequency response centered at 500 kHz with a 60% or greater bandwidth about that center frequency, whereas amplifier 86 may have a center frequency of 500 kHz with a 30% bandwidth or less about that center frequency. It will be appreciated that the function of these separate amplifiers may be implemented by changing a filtering or tuning of an individual amplifier. Repetitive measurements may be made to further reduce signal-to-noise in these measurements and filtering may be applied.

The data collected from the array ultrasound transducer 34 may be amplified, for example, with an 86 dB amplification and collected by a 12-bit analog-to-digital converter at 80 MHz sampling rate. The resulting QUS and PA signals are received by the controller 70 which may extract time-of-flight, broadband ultrasound attenuation, and frequency-dependent PA information.

A rotary coupler 88, for example, using a spring retracted electrical cable, allows communication between the controller 70 and a user workstation 90 in either rotated position of the housing 12. Wireless communication may also be used to provide for communication between the controller 70 and the user workstation 90.

Referring again to FIGS. 1-4, all surfaces defining the cavity 14 are treated to absorb excess laser light energy through the use of absorbing pigment and/or surface roughness. Generally these treatments reduce reflectivity of the emitted laser light to less than 20% and desirably less than 15%. Baffle channels may also be employed to trap light reflected from the skin so as to minimize the possibility that this light will stimulate ultrasound from tissue.

Similarly, the walls of the cavity 14 may be broken with sound blocking air gaps or elastomeric material to dampen the conduction of ultrasound through the structure of the housing.

The ultrasonic signal measurements may be processed by the ultrasound bone densitometer or the workstation 90 to produce an image and various quantitative measurements provided in graphical form on the display of the workstation 90. The unprocessed time domain waveforms may also be displayed for verification purposes.

Generally the processing of the PA signals may use spectral unmixing based on the least-square regression method to separate signals from different tissue. The four chromophores considered in spectral unmixing are deoxy-hemoglobin, oxy-hemoglobin, lipid, and hydroxyapatite. By performing spectral unmixing, the relative contents of the chemical components of the two groups (control vs. osteoporosis) are obtained. The relative content of each optically absorbing chemical component derived from the spectral unmixing reflects the weight of this chemical component in the PA-measured optical absorption spectrum of the bone.

In trabecular bones, both the microstructure and the ultrasound attenuation affect PA spectral analysis. The PA signals generated at different locations and at different frequencies are first compensated for by ultrasound attenuation. The PA spectral analysis of bone micro-architecture is performed at two laser wavelengths (800 nm and 930 nm). The 800-nm wavelength is the isosbestic point for the optical absorption of oxy- and deoxy-hemoglobin and thus is used to present the content or spatial distribution of whole blood. In contrast, the 930-nm wavelength corresponds to the strong optical absorptions of lipid. Inter-trabecular pores are filled with bone marrow rich in both blood and lipid clusters, and hence the measurements from PA spectral analyses at these two wavelengths are used together to reflect the heterogeneous spatial distributions of lipid and blood in trabecular bone as well as trabecular porosity.

With radio-frequency (RF) PA signals from each trabecular site acquired, the power spectral density (PSD) is derived. Generally osteoporosis will be associated with lower high-frequency components in comparison with the non-osteoporotic individuals. This may be due to the fact that the osteoporosis bone has larger porosity, which is filled by marrow, and the spatially distributed marrow with larger scales generates PA signals with lower frequency.

When combined with the spectral unmixing methods, multi-wavelength PA measurements of a bone can evaluate the contents of not only non-organic minerals (hydroxyapatite) but also organic materials such as lipid and hemoglobin. Hence, clinically significant bone physiological properties that have been the targets of MRI and MRS, including bone fat content and perfusion, can be quantitatively assessed by PA measurement with a comparable sensitivity but at a much lower cost.

By quantitatively analyzing the power spectra of the radio-frequency (RF) PA signals from the calcaneus, the micro-features in the trabecular bone can be evaluated. By the nature of PA measurement, the frequency of sound generated by the laser is directly a function of the size of the optically absorbing micro-structures. By working at different laser wavelengths that are preferentially absorbed by different chemical contents in the bone, the present invention can evaluate the heterogeneous distributions of different organic and non-organic substances.

The PA measurement is a natural complement to the existing QUS bone assessment devices. By detecting the spectroscopic optical absorption in the bone, PA measurement can probe bone molecular and chemical information which is highly valuable for assessing the physiology and underlying pathology in the bone. The existing QUS allows the measurements of the additional parameters of BUA, SOS, and stiffness, reflecting different aspects of bone health which may work together for describing early disease processes, evaluating early treatment response, and predicting treatment outcome. Once translated to bone health clinics, such a device would facilitate early treatment modification and personalized medicine, changing the landscape of osteoporosis management.

Generally, the processing of the signals may include normalization to calibration obtained, for example, using phantoms placed in lieu of the foot 16 and calibration measurements of laser energy, for example, using a beam splitter and laser energy meter. Laser power may be adjusted in real time by a feedback loop measuring received acoustic energy at the array ultrasound transducer 34. The length of light pulse duration may be optimized to improve generation of 0.5-2.0 MHz ultrasound.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a controller” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. An ultrasound bone densitometer comprising:
a first and second laterally opposed ultrasound transducer for transmitting an ultrasound signal through a bone;
a light source movable between a first position in a region between the bone and the second opposed ultrasound transducer and a second position displaced from the region between the bone and the second opposed ultrasound transducer; the light source operating in the first position to direct the light beam along an axis through the bone; and
a controller for controlling movement of the light source and activation of the light source and second ultrasound transducer and for collecting data from the first ultrasound transducer.

2. The ultrasound bone densitometer of claim 1 wherein light source is a diverter receiving off-axis light from a laser to divert the light along the axis laterally through the bone.

3. The ultrasound bone densitometer of claim 2 wherein the diverter is a front surface mirror sealed within a liquid tight housing to redirect the light beam through a sealed cavity window through the bone.

4. The ultrasound bone densitometer of claim 1 further including first and second inflatable coupling membranes positioned between the first and second opposed transducers and respective sides of the bone wherein the inflatable coupling membrane for the second opposed ultrasound transducer is controllably deflated by the controller to permit movement of the light source to the first position.

5. The ultrasound bone densitometer of claim 4 further including an ankle stop positioned around the second inflatable coupling membrane to support the ankle against lateral movement when the inflatable coupling for the second opposed ultrasound transducer is deflated.

6. The ultrasound bone densitometer of claim 1 further including a housing providing a cavity for receiving and supporting a patient foot therein with the bone positioned between first and second laterally opposed ultrasound transducers at either of two positions having a respective 180° rotation about a vertical axis.

7. The ultrasound bone densitometer of claim 1 further including a housing providing a cavity for receiving and supporting a patient foot therein with the bone positioned between first and second laterally opposed ultrasound transducers wherein inner surfaces of the cavity provide a reflectivity of less 20% in a range of 690-950 nm.

8. The ultrasound bone densitometer of claim 1 further including a housing providing a cavity for receiving and supporting a patient foot therein with the bone positioned between first and second laterally opposed ultrasound transducers and including a light blocking cover fitting over the cavity during activation of the light source.

9. The ultrasound bone densitometer of claim 8 wherein the light blocking cover is an inflatable light baffle extended over the bone of a foot by inflation.

10. The ultrasound bone densitometer of claim 1 wherein including a first and second amplification path for receiving signals from the first ultrasound transducer, the first amplifier path receiving signals during activation of the second ultrasound transducer and the second amplifier path receiving signals during activation of the light source; wherein the first amplifier path provides a greater bandwidth than the second amplifier path.

11. The ultrasound bone densitometer of claim 1 wherein the controller operates to measure ultrasound signals produced by the light source and captured by the first ultrasound transducer when the light source is both directed from a left to right direction and right to left direction.

12. The ultrasound bone densitometer of claim 1 wherein the controller operates to measure a time-of-flight of acoustic energy through the bone, a broadband ultrasound attenuation of ultrasound to the bone, and a frequency-dependent photo acoustic signal produced by the light source.

13. An ultrasound bone densitometer comprising:
a first and second laterally opposed ultrasound transducer for transmitting an ultrasound signal through a bone to make a first ultrasound measurement;
a light source operating to direct the light beam along an axis in a first direction through the bone at a first time to make a second ultrasound measurement and to direct the light beam along the axis in a second direction through the bone at a second time to make a third ultrasound measurement; and
a controller for combining the ultrasound measurements to provide an output.

14. An ultrasound bone densitometer comprising:
housing providing a cavity for receiving and supporting a patient foot therein;
a first and second laterally opposed ultrasound transducer for transmitting an ultrasound signal through a bone in the patient's foot;
a light source positioned to direct the light beam along an axis through the bone; and
a controller for controlling movement of the light source and activation of the light source and second ultrasound transducer and for collecting data from the first ultrasound transducer,
wherein inner surfaces of the cavity provide a reflectivity of less 20% in a range of 690-950 nm.

15. An ultrasound bone densitometer comprising:
a first and second laterally opposed ultrasound transducer for transmitting an ultrasound signal through a bone;
a light source operating to direct the light beam along an axis through the bone; and
a controller for controlling movement of the light source and activation of the light source and second ultrasound transducer and for collecting data from the first ultrasound transducer; and
further including a housing providing a turntable supporting a cavity for receiving and supporting a patient foot therein with the bone positioned between first and second laterally opposed ultrasound transducers at either of two positions having a respective 180° rotation about a vertical axis with the housing rotated on the turntable.
